(19) United States
(12) Patent Application Publication
(10) Pub. No.: US 2023/0072835 A1
(43) Pub. Date: Mar. 9, 2023

(54) Recombinant microorganism for producing L-valine, construction method and application thereof

(71) Applicants: ANHUI HUAHENG BIOTECHNOLOGY CO., LTD., Hefei, Anhui (CN); TIANJIN INSTITUTE OF INDUSTRIAL BIOTECHNOLOGY CHINESE ACADEMY OF SCIENCES, Tianjin (CN)

(72) Inventors: Xueli ZHANG, Tianjin (CN); Henghua GUO, Hefei, Anhui (CN); Pingping LIU, Tianjin (CN); Dongzhu ZHANG, Hefei, Anhui (CN); Jinlei TANG, Tianjin (CN); Chengxiu HAN, Bayannaoer, Inner Mongolia (CN); Siqing TANG, Bayannaoer, Inner Mongolia (CN); Shupeng LIU, Hefei, Anhui (CN); Yanhe MA, Tianjin (CN)

(21) Appl. No.: 17/603,008

(22) PCT Filed: Dec. 18, 2020
(86) PCT No.: PCT/CN2020/137780

§ 371 Date: Oct. 12, 2021

(30) Foreign Application Priority Data
May 13, 2020 (CN) 202010401422.5

(51) Int. Cl. C12P 13/08; C12N 9/06; C12N 9/02; C12N 9/12; C12N 15/52; C12N 15/90

(52) U.S. Cl. CPC C12P 13/08; C12N 9/0016; C12Y 104/01009; C12N 9/0036; C12Y 106/01002; C12N 9/1205; C12Y 207/01023; C12N 15/52; C12N 15/902

(57) ABSTRACT

Related are a recombinant microorganism for producing L-valine, a construction method and an application thereof. Through transferring an acetohydroxy acid reductoisomerase gene and/or an amino acid dehydrogenase gene into a microorganism, and enhancing activity of an acetohydroxy acid reductoisomerase and/or an amino acid dehydrogenase, the titer and yield of L-valine generated by Escherichia coli may be improved, and L-valine was produced by one-step anaerobic fermentation.

### CROSS-REFERENCE TO RELATED APPLICATION

The present application is a National Stage of International Patent Application No: PCT/CN2020/137780 filed on 18 Dec. 2020, which claims the benefit of priority to Chinese Patent Application No. 202010401422.5 filed to the China National Intellectual Property Administration on 13 May 2020 and entitled “Recombinant microorganism for producing L-valine, construction method and application thereof”, the content of which is hereby incorporated by reference in its entirety.

### SEQUENCE LISTING

The present application contains a Sequence Listing which has been submitted electronically in ASCII format and is hereby incorporated by reference in its entirety. Said ASCII copy is named_Sequence_Listing.txt and is 16.7 kilobytes in size, and contains 70 sequences from SEQ ID NO:1 to SEQ ID NO:70, which are identical to the sequence listing filed in the corresponding international application No. PCT/CN2020/137780 filed on 18 Dec. 2020.

### TECHNICAL FIELD

The disclosure relates to a construction method of a recombinant microorganism for producing L-valine, the recombinant microorganism obtained by the construction method, specifically recombinant Escherichia coli, and a method for producing L-valine through a fermentation method.

### BACKGROUND

L-valine, as one of three branched-chain amino acids (BCAA), is an essential amino acid. It cannot be synthesized in humans and animals, and can only be acquired by supplementation in vitro. Nowadays, L-valine has been widely used in the fields of food and medicine, mainly including food additive, nutritional supplement and flavoring agent and the like; and widely used in preparation of cosmetics, as precursors of antibiotics or herbicides and the like. Additionally, along with the increasing demands for feed quality and ratio, the role of L-valine in the feed additive industry will become more and more important in the future. And this will result the expand of its demand and great potential in the future market.

L-valine can be directly synthesized by microbial cells. However, the production capacity of wild-type cells is greatly limited by the intracellular regulatory network, such as feedback inhibition and so on. To obtain a strain capable of efficiently producing L-valine by fermentation process, these self-regulatory mechanisms of the microbial cells must be effectively eliminated. At present, L-valine is mainly produced through fermentation method in the world. Strains used for fermentative production of L-valine are mostly obtained by mutagenesis, and original strains mainly include Corynebacterium glutamicum, Brevibacterium flavum and the like. For example, starting from a Brevibacterium flavum, Ning Chen et al. produced mutant strains by using protoplast ultraviolet mutagenesis in combination with the DES chemical mutagenesis. Finally, an efficiently L-valine-producing strain TV2564 was screened and selected. But a notable fact is that strains obtained by traditional mutagenesis have many limits, such as strong randomness, unclear genetic background, producing by-products during fermentation, and not easy to modified for obtaining much more highly efficient strains.

With the rapid development of synthetic biology and metabolic engineering in recent years, recombinant engineered strains have been developed and achieved good results. These strains can efficiently produce L-valine, have clear genetic background, and are easy to cultivated. In 2007, Sang Yup Lee's group developed an L-valine-producing strain starting from the Escherichia coli W3110 by in combination of different strategies, such as the rational metabolic engineering, transcriptome analysis and genetic modification, and gene knockout. This strain can produce L-valine in batch culture under aerobic conditions. But it needs continuous oxygen supply during the culture process and L-isoleucine needs to be added to ensure the normal growth.

Xie xixian et al. integrate an encoding gene alsS of Bacillus subtilis acetolactate synthase to release the feedback inhibition of L-valine on a synthetic pathway, and integrate a mutant gene spoTM of Escherichia coli ppGpp3′-pyrophosphohydrolase to enhance supply of a pyruvate, and enhance a level of L-valine generated by shake flask fermentation of an original strain VHY03.

Most of the publicly reported L-valine fermentation production is achieved through aerobic or two-step fermentation at present. However, the aerobic process requires air during a production process and consumes a lot of energy; more critically, a considerable part of a carbon source enters a tricarboxylic acid cycle (TCA) and is consumed by cell growth resulting that the yield of L-valine is always much lower than the theoretical maximum. Compared with the aerobic process, the anaerobic process has advantages of low energy consumption and high yield of L-valine. The air is not needed during a production process, and the energy is greatly saved; and a yield of L-valine is usually close to the theoretical maximum. The anaerobic fermentation of amino acid production is firstly achieved in a process of alanine production. At present, there are no reports of pure anaerobic fermentation processes for production of other amino acids.

In addition, the overexpression of key genes involved in the metabolic engineering of L-valine strains are always plasmid-borne, so that antibiotics need to be added to maintain the existence of the plasmid during the fermentation process, a production cost is increased and a risk of plasmid loss exists in industrial production.

Therefore, there is still a need in the field to provide high-yield, energy-saving, simple and stable recombinant microorganisms for producing L-valine and a corresponding production and preparation method for L-valine.

### SUMMARY

It is discovered in the disclosure that through enhancing activity of an acetohydroxy acid isoreductase and/or an amino acid dehydrogenase, titer and yield of L-valine generated by Escherichia coli can be improved, and one-step anaerobic fermentation of L-valine can be achieved.

The first aspect of the disclosure is to provide a construction method for construction of a recombinant microorganism producing L-valine. The recombinant microorganism obtained by this method has stable genetic background and balanced L-valine reducing power, and is suitable for one-step anaerobic fermentation.

In the present disclosure, “enhancement” of enzyme activity refers to enhancement of intracellular activity of one or more enzymes which has corresponding gene in the microogranism. The enhancement of the activity may be achieved by any suitable methods known in the field, for example, by overexpression, it includes but not limited to increasing the copy number of the gene or allele, modifying a nucleotide sequence for guiding or controlling gene expression, using a strong promoter to increase protein activity or concentration by 10%-500% compared to an initial microbial level.

In one embodiment, an acetohydroxy acid isoreductase gene or/and an amino acid dehydrogenase gene are transferred into a microorganism, so that the enzyme activity is enhanced.

In one embodiment, the acetohydroxy acid isoreductase gene and the amino acid dehydrogenase gene transferred in the disclosure are exogenous to the microorganism transferred. The acetohydroxy acid isoreductase gene and the amino acid dehydrogenase gene may be a corresponding gene from any microorganisms such as Lactococcus, and Bacillus.

In one embodiment, the acetohydroxy acid isoreductase gene and/or amino acid dehydrogenase gene are NADH-dependent.

Most of reducing power types of cell generation under anaerobic conditions is NADH. In order to achieve high-efficiency generation of L-valine under anaerobic conditions, the problem of cofactor imbalance must be solved. In one embodiment of the disclosure, the NADH-dependent acetohydroxy acid isoreductase gene and/or amino acid dehydrogenase gene may be selected to consume excessive NADH under anaerobic conditions and solve the problem of reducing power imbalance during the anaerobic fermentation.

In one embodiment, the acetohydroxy acid isoreductase gene is ilvC or KARI; and the amino acid dehydrogenase gene is a leucine dehydrogenase gene.

In one preferred embodiment, the acetohydroxy acid isoreductase gene is KARI, and the leucine dehydrogenase gene is leuDH.

The acetohydroxy acid isoreductase gene and the amino acid dehydrogenase gene may exist in the microorganism in any suitable forms known in the field, for example, in the form of a plasmid, or the form of being integrated into a genome. In one embodiment, the enzyme encoding gene integrated into the genome is placed under the control of a suitable regulatory element.

The regulatory element is selected from an M1-46 artificial regulatory element, an M1-93 artificial regulatory element or a RBS5 artificial regulatory element.

In one embodiment, the M1-46 artificial regulatory element regulates an ilvC gene.

In one embodiment, the M1-93 artificial regulatory element regulates a leuDH gene;

In one embodiment, the RBS5 artificial regulatory element regulates a KARI gene.

In one embodiment, the disclosure further includes the following modifications to one or more of the following enzyme genes of the above recombinant microorganism, so that the activity of these enzymes is reduced or inactivated.

(1) Knocking out a methylglyoxal synthase (mgsA) gene;

(2) knocking out a lactate dehydrogenase (ldhA) gene;

(3) knocking out phosphoacetyl transferase (pta) and/or acetate kinase (ackA) genes;

(4) knocking out propionate kinase (tdcD) and/or formate acetyltransferase (tdcE) genes;

(5) knocking out an alcohol dehydrogenase (adhE) gene; and

(6) knocking out fumarate reductase (frd) and/or pyruvate formate lyase (pflB) genes.

It may be understood by those skilled in the art that gene knockout may be performed in a manner known in the prior art, so that the activity of the enzyme is reduced or inactivated. The knockout operation is aimed at an endogenous enzyme gene of the original microorganism, so that the above endogenous enzyme activity of the microorganism is reduced or inactivated.

An encoding sequence of the enzyme gene in the above (1)-(6) may also be substituted with an encoding sequence of another gene by means of genetic engineering such as homologous recombination, thereby the above endogenous enzyme activity of the microorganism is reduced or inactivated. The gene to substitute these endogenous enzymes may be a gene to be enhanced for expression, such as the above ilvC gene, KARI gene or leuDH gene.

In one embodiment, it further includes enhancing activity of an acetolactate synthase (AHAS) and/or a dihydroxy acid dehydratase (ilvD) in the recombinant microorganism of the disclosure.

In one preferred embodiment, the AHAS is selected from ilvBN, ilvGM or ilvIH, and the activity of at least one of them is enhanced.

In one preferred embodiment, the activity of the AHAS is enhanced by releasing feedback inhibition of a valine to ilvIH, for example, the feedback inhibition of the valine to the ilvIH is released by mutating an ilvH gene.

For the involved acetolactate synthase used in biologically synthesizing L-valine, in addition to an isozyme II (herein also referred to as AHAS II), an isozyme III (herein also referred to as AHAS III) is also known. The AHASIII is encoded by an ilvIH operon, and the operon is formed by ilvI encoding a large subunit (catalytic subunit) and ilvH encoding a small subunit (control subunit). The AHASIII is feedback inhibited by L-valine. A reported method may be used to mutate the ilvI gene, such as amino acid substitution of ilvH 14 Gly→Asp (Vyazmensky, M. et al., “Biochemistry” 35:10339-10346 (1996)) and/or ilvH 17Ser→Phe (U.S. Pat. No. 6,737,255B2); and ilvH612 (De Felice et al., “Journal of Bacteriology” 120:1058-1067 (1974)) and the like.

In one embodiment, activity of a dihydroxy acid dehydratase (ilvD) in the recombinant microorganism of the disclosure is enhanced, for example, the ilvD gene is transferred into the microorganism to enhance the activity of the ilvD.

The activity of the AHAS and/or the dihydroxy acid dehydratase (ilvD) is enhanced, and it is operated optionally in combination with any one or more of the above modifications (1)-(6).

In one embodiment, the operation is performed in combination with the modification of the item (2).

In one embodiment, the operation is performed in combination with the modification of the item (6).

In one embodiment, the operation is performed in combination with the modifications of the item (2) and the item (5).

In one embodiment, the operation is performed in combination with the modifications of the items (1) and (3)-(6).

In one embodiment, the operation is performed in combination with the modifications of the items

In one embodiment, optionally, the knockout of the item (1) is achieved by substituting the endogenous mgsA gene of the microorganism with the ilvC gene.

In one embodiment, the knockout of the item (6) is achieved by substituting the endogenous pflB gene of the microorganism with the ilvD gene, and/or substituting the endogenous frd gene of the microorganism with the leuDH gene.

In one embodiment, the knockout of the item (5) is achieved by substituting the endogenous adhE gene of the microorganism with the KARI gene.

The substitution may be that, in a mode known to those skilled in the art, an encoding sequence of a gene to be inserted is integrated into a substituted gene in the microbial chromosome, so that the gene encoding sequence in the original site is substituted with the encoding sequence of the integrated inserted gene.

Preferably, the substitution of the KARI, ilvC, ilvD and leuDH occur simultaneously, herein the ilvC gene is optionally knocked out again.

In one embodiment, the microorganism is Escherichia coli.

In one embodiment, the microorganism is Escherichia coli ATCC 8739.

In one embodiment, at least one regulatory element is used to regulate the genes encoding the above enzymes involved.

In one embodiment, the regulatory element is selected from an M1-46 artificial regulatory element, an M1-93 artificial regulatory element or a RBS5 artificial regulatory element.

In one embodiment, the M1-46 artificial regulatory element regulates an ilvC gene.

In one embodiment, the M1-93 artificial regulatory element regulates the ilvD, leuDH, ilvBN and ilvGM genes.

In one embodiment, the RBS5 artificial regulatory element regulates a KARI gene.

The regulatory element may be inserted at an upstream of the ilvC gene by a known genetic engineering method. The method includes, but is not limited to, inserting the sequence of the regulatory element into the upstream of the gene encoding sequence of the target enzyme by means of gene recombination, for example, by means of homologous recombination, so as to enhance the intensity of the target gene expression.

In one embodiment, herein the enzyme encoding gene and the regulatory element are integrated into the genome of the microorganism.

In one embodiment, a plasmid containing the enzyme encoding gene and the regulatory element sequence is transferred into the microorganism.

In one embodiment, the transfer, mutation or knockout of the target enzyme gene is completed by a method of integrating into the genome of the microorganism.

In one embodiment, the transfer, mutation or knockout of the enzyme gene is completed by a method of homologous recombination.

In one embodiment, the transfer, mutation or knockout of the enzyme gene is completed by a method of two-step homologous recombination.

The homologous recombination may be implemented by the homologous recombination system known to the related art, such as an Escherichia coli RecA recombination system or a Red recombination system, to transfer, mutate or knock out the target gene.

The two-step homologous recombination method to transfer, mutate or knock out the target gene includes the following steps (Escherichia coli are taken as an example):

(1) Preparation of a DNA fragment I: a pXZ-CS plasmid (Tan, et al., Appl Environ Microbiol, 2013, 79:4838-4844) DNA is used as a template, an amplification primer 1 is used to amplify the DNA fragment I, and used for the first step of homologous recombination;

(2) the first step of the homologous recombination: a pKD46 plasmid (Datsenko and Wanner 2000, Proc Natl Acad Sci USA 97:6640-6645) is transformed into Escherichia coli, and then the DNA fragment I is transformed into the Escherichia coli containing pKD46, a detection primer 1 is used to verify the transformed bacteria and select the correct colony;

(3) preparation of a DNA fragment II: the original Escherichia coli are used as a template, and an amplification primer 2 is used to amplify the DNA fragment II. The DNA fragment II is used for a second step of homologous recombination; and

(4) the second step of the homologous recombination: the DNA fragment II is transformed into the strain obtained in the second step; and a detection primer 2 is used to verify the transformed bacteria and select a correct colony.

The second aspect of the disclosure provides a recombinant microorganism for producing L-valine obtained by using the above construction method, specifically recombinant Escherichia coli, it contains an acetohydroxy acid isoreductase and/or an amino acid dehydrogenase gene,

In one embodiment, the Escherichia coli ATCC 8739 is used as an original strain, and coupling of intracellular cofactor NADH supply and cell growth is achieved through the gene homologous recombination, thereby coupling (FIG. 1) of the cell growth and L-valine production under anaerobic conditions is achieved.

In one embodiment, the recombinant Escherichia coli obtained by the above construction method undergo metabolic evolution, for example, through 50 generations, 70 generations, 80 generations, 90 generations, 100 generations, and 120 generations, the recombinant Escherichia coli for highly producing L-valine are obtained. In one embodiment, a recombinant Escherichia coli strain producing L-valine is obtained after 105 generations of metabolic evolution. It is preserved in China General Microbiological Culture Collection Center (CGMCC) on Mar. 6, 2020, the Depository Authority is located at No. 3, Courtyard 1, Beichen West Road, Chaoyang District, Beijing, the preservation number is: CGMCC 19458, and the classification name is Escherichia coli.

The third aspect of the disclosure is an application of the recombinant microorganism obtained by the above method in producing L-valine.

The fourth aspect of the disclosure is a method for fermentatively producing L-valine using the recombinant microorganism obtained by the above construction method, including: (1) fermenting the recombinant microorganism obtained by the above construction method; and (2) separating and harvesting L-valine.

In one embodiment, the fermentation is anaerobic fermentation.

In one embodiment, the anaerobic fermentation includes the following steps:

(1) seed culture: a clone on a plate is picked and inoculated into a seed culture medium at 37° C., and shake culture is performed, to obtain seed culture solution; and

(2) fermentation culture: the seed culture solution is inoculated into fermentation culture medium, and culturing at 37° C. and 150 rpm for 4 days, to obtain fermentation solution. The pH during fermentation process is controlled at 7.0. No air was sparged during the fermentation.

Herein the seed culture medium is formed by the following components (a solvent is water):

Glucose 20 g/L, corn syrup dry powder 10 g/L, KH₂PO₄ 8.8 g/L, (NH₄)₂SO₄ 2.5 g/L, and MgSO₄.7H₂O 2 g/L.

The fermentation culture medium and the seed culture medium have the same components, and a difference is only that the glucose concentration is 50 g/L.

The beneficial effects of the disclosure:

(1) Compared with previous production methods and strains, the disclosure achieves the one-step anaerobic fermentation production of L-valine, reduces the production cost and improves the yield of L-valine.

(2) The disclosure preferably constructs a genetically stable L-valine production strain by modified directly on the genome of the recombinant microorganism rather than in the form of plasmid, and additional addition of substances such as antibiotics and inducers does not require, and the production process is stable and easy to operate.

(3) Through metabolic evolution, the titer and yield of L-valine and cell tolerance are improved significantly in the recombinant microorganisms.

Preservation of Biological Material

Recombinant Escherichia coli Sval065 constructed in the disclosure are classified and named:

Escherichia coli. A biological material is submitted for preservation on Mar. 6, 2020, Depository Authority: China General Microbiological Culture Collection Center (CGMCC), the preservation address is No. 3, Courtyard 1, Beichen West Road, Chaoyang District, Beijing, Telephone: 010-64807355, and the preservation number is CGMCC No. 19458.

### BRIEF DESCRIPTION OF THE DRAWINGS

Drawings of the description for constituting a part of the present disclosure are used to provide further understanding of the disclosure. Exemplary embodiments of the disclosure and descriptions thereof are used to explain the disclosure, and do not constitute improper limitation to the disclosure. In the drawings:

FIG. 1: L-valine synthesis pathway.

FIG. 2: Determination of a standard substance of L-valine measured by high performance liquid chromatography.

FIG. 3: Determination of fermentation solution components of strain Sval064 by high performance liquid chromatography.

FIG. 4: Construction of the strain Sval065 by metabolic evolution.

FIG. 5: Determination of a standard substance of L-valine by high performance liquid chromatography.

FIG. 6: Determination of fermentation solution components of strain Sval065 by high performance liquid chromatography.

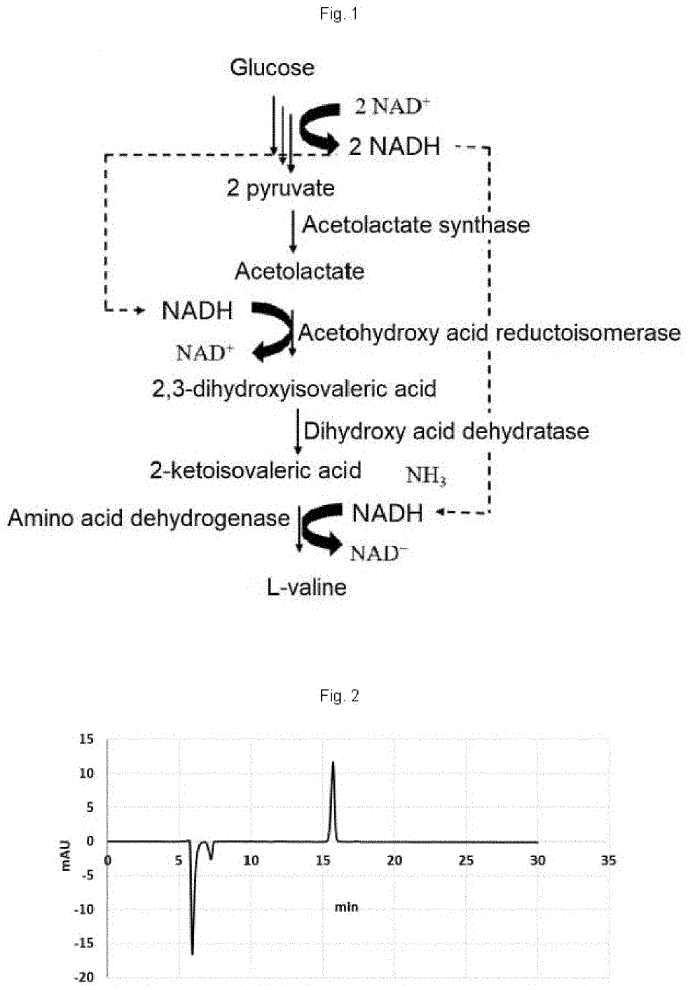

### DETAILED DESCRIPTION OF THE EMBODIMENTS

The disclosure is further described by the following embodiments, but any embodiments or combinations thereof should not be interpreted as limiting a scope or an embodiment of the disclosure. The scope of the disclosure is defined by appended claims. In combination with the description and common knowledge in the field, those of ordinary skill in the art may clearly understand the scope defined by the claims. Without departing from the spirit and scope of the disclosure, those skilled in the art may make any modifications or changes to technical schemes of the disclosure, and such modifications and changes are also included in the scope of the disclosure.

Experimental methods used in the following embodiments are conventional methods unless otherwise specified. Materials, reagents and the like used in the following embodiments may be obtained from commercial sources unless otherwise specified.

Strains and plasmids constructed in this research are shown in Table 1, and primers used are shown in Table 2.

TABLE 1
Strains and plasmids used in the disclosure

| | Related characteristics | Sources |
|---|---|---|
| Strain | | |
| ATCC 8739 | Wild type | Laboratory |
| | | preservation |
| M1-93 | ATCC 8739, | Lu, et al., Appl |
| | FRT-Km-FRT::M1-93::lacZ | Microbiol |
| | | Biotechnol, 2012, |
| | | 93:2455-2462 |
| M1-46 | ATCC 8739, | Lu, et al., Appl |
| | FRT-Km-FRT::M1-46::lacZ | Microbiol |
| | | Biotechnol, 2012, |
| | | 93:2455-2462 |
| Sval001 | ATCC 8739, mgsA::cat-sacB | Constructed by the |
| | | disclosure |
| Sval002 | Sval001, ΔmgsA | Constructed by the |
| | | disclosure |
| Sval003 | Sval002, ldhA::cat-sacB | Constructed by the |
| | | disclosure |
| Sval004 | Sval003, ΔldhA | Constructed by the |
| | | disclosure |
| Sval005 | Sval004, ackA-pta::cat-sacB | Constructed by the |
| | | disclosure |
| Sval006 | Sval005, Δ ackA-pta | Constructed by the |
| | | disclosure |
| Sval007 | Sval006, tdcDE::cat-sacB | Constructed by the |
| | | disclosure |
| Sval008 | Sval007, ΔtdcDE | Constructed by the |
| | | disclosure |
| Sval009 | Sval008, adhE::cat-sacB | Constructed by the |
| | | disclosure |
| Sval010 | Sval009, ΔadhE | Constructed by the |
| | | disclosure |
| Sval011 | Sval010, mgsA::cat-sacB | Constructed by the |
| | | disclosure |
| Sval012 | Sval011, mgsA::ilvC | Constructed by the |
| | | disclosure |
| Sval013 | Sval012, mgsA::cat-sacB::ilvC | Constructed by the |
| | | disclosure |
| Sval014 | Sval013, mgsA::M1-46-ilvC | Constructed by the |
| | | disclosure |
| Sval015 | Sval014, pflB::cat-sacB | Constructed by the |
| | | disclosure |
| Sval016 | Sval015, pflB::ilvD | Constructed by the |
| | | disclosure |
| Sval017 | Sval016, pflB::cat-sacB::ilvD | Constructed by the |
| | | disclosure |
| Sval018 | Sval017, pflB::RBS4-ilvD | Constructed by the |
| | | disclosure |
| Sval019 | Sval018, cat-sacB::ilvB | Constructed by the |
| | | disclosure |
| Sval020 | Sval019, M1-93:: ilvB | Constructed by the |
| | | disclosure |
| Sval021 | Sval020, cat-sacB::ilvG | Constructed by the |
| | | disclosure |
| Sval022 | Sval021, M1-93:: ilvG | Constructed by the |
| | | disclosure |
| Sval023 | Sval022, ilvH::cat-sacB | Constructed by the |
| | | disclosure |
| Sval024 | Sval023, ilvH:: ilvH* | Constructed by the |
| | | disclosure |
| Sval025 | Sval024, frd::cat-sacB | Constructed by the |
| | | disclosure |
| Sval026 | Sval025, frd::M1-93-leuDH | Constructed by the |
| | | disclosure |
| Sval061 | Sval026, adhE::cat-sacB | Constructed by the |
| | | disclosure |
| Sval062 | Sval061, adhE::RBS5-kari | Constructed by the |
| | | disclosure |
| Sval063 | Sval062, mgsA::cat-sacB | Constructed by the |
| | | disclosure |
| Sval064 | Sval063, ΔmgsA | Constructed by the |
| | | disclosure |
| Sval065 | metabolic evolution of | Constructed by the |
| | Sval064 for 105 generations | disclosure |
| Plasmid | | |
| pUC57-M1-93-leuDH | Artificial regulatory element | Nanjing Genscript |
| | M1-93 and chemically | Biotechnology |
| | synthesized gene leuDH are | Co., Ltd. |
| | linked to a pUC57 vector | |
| | together | |
| pUC57-RBS5-kari | Artificial regulatory element | Nanjing Genscript |
| | RBS5 and chemically | Biotechnology |
| | synthesized gene kari are | Co., Ltd. |
| | linked to the pUC57 vector | |
| | together | |

TABLE 2
Primers used in the disclosure

| Primer | | Sequence |
|---|---|---|
| name | Sequence | number |
| mgsA-cs-up | gtaggaaagttaactacggatgtacattatggaactgacgactcgcactt | 1 |
| | TGTGACGGAAGATCACTTCGCAG | |
| mgsA-cs-down | gcgtttgccacctgtgcaatattacttcagacggtccgcgagataacgctT | 2 |
| | TATTTGTTAACTGTTAATTGTCCT | |
| XZ-mgsA-up | cagctcatcaaccaggtcaa | 3 |
| XZ-mgsA-down | aaaagccgtcacgttattgg | 4 |
| mgsA-del-down | gcgtttgccacctgtgcaatattacttcagacggtccgcgagataacgcta | 5 |
| | agtgcgagtcgtcagttcc | |
| mgsA-ilvC-up | gtaggaaagttaactacggatgtacattatggaactgacgactcgcactt | 6 |
| | ATGGCTAACTACTTCAATACac | |
| mgsA-ilvC-down | gcgtttgccacctgtgcaatattacttcagacggtccgcgagataacgctT | 7 |
| | TAACCCGCAACAGCAATACGtttc | |
| mgsA-Pcs-up | gtaggaaagttaactacggatgtacattatggaactgacgactcgcactt | 8 |
| | TGTGACGGAAGATCACTTCGCAG | |
| mgsA-Pcs-down | agctgtgccagctgctggcgcagattcagtGTATTGAAGTAGTTA | 9 |
| | GCCATTTATTTGTTAACTGTTAATTGTCCT | |
| mgsA-P46-up | gtaggaaagttaactacggatgtacattatggaactgacgactcgcactt | 10 |
| | TTATCTCTGGCGGTGTTGAC | |
| ilvC-P46-down | agctgtgccagctgctggcgcagattcagtGTATTGAAGTAGTTA | 11 |
| | GCCATAGCTGTTTCCTGGTTTAAACCG | |
| ilvC-YZ347-down | cgcacta catcagagtgctg | 12 |
| ldhA-cs-up | ttcaacatcactggagaaagtcttatgaaactcgccgtttatagcacaaa | 13 |
| | TGTGACGGAAGATCACTTCGCAG | |
| ldhA-cs-down | agcggcaagattaaaccagttcgttcgggcaggtttcgcctttttccagaT | 14 |
| | TATTTGTTAACTGTTAATTGTCCT | |
| XZ-ldhA-up | GATAACGGAGATCGGGAATG | 15 |
| XZ- ldhA-down | CTTTGGCTGTCAGTTCACCA | 16 |
| ldhA-del-down | agcggcaagattaaaccagttcgttcgggcaggtttcgcctttttccagattt | 17 |
| | gtgctataaacggcgagt | |
| ackA-cs-up | aggtacttccatgtcgagtaagttagtactggttctgaactgcggtagttTG | 18 |
| | TGACGGAAGATCACTTCGCAG | |
| pta-cs-down | ggtcggcagaacgctgtaccgctttgtaggtggtgttaccggtgttcagaT | 19 |
| | TATTTGTTAACTGTTAATTGTCCT | |
| XZ-ackA-up | cgggacaacgttcaaaacat | 20 |
| XZ-pta-down | attgcccatcttcttgttgg | 21 |
| ackA-del-down | ggtcggcagaacgctgtaccgctttgtaggtggtgttaccggtgttcagaa | 22 |
| | actaccgcagttcagaacca | |
| tdcDE-cs-up | ccgtgattggtctgctgaccatcctgaacatcgtatacaaactgttttaaTG | 23 |
| | TGACGGAAGATCACTTCGCAG | |
| tdcDE-cs-down | cgcctggggcacgttgcgtttcgataatctttttcatacatcctccggcgTT | 24 |
| | ATTTGTTAACTGTTAATTGTCCT | |
| XZ-tdcDE-up | TGATGAGCTACCTGGTATGGC | 25 |
| XZ-tdcDE-down | CGCCGACAGAGTAATAGGTTTTAC | 26 |
| tdcDE-del-down | cgcctggggcacgttgcgtttcgataatctttttcatacatcctccggcgtt | 27 |
| | aaaacagtttgtataegatgttcag | |
| adhE-cs-up | ATAACTCTAATGTTTAAACTCTTTTAGTAAATCACAG | 28 |
| | TGAGTGTGAGCGCTGTGACGGAAGATCACTTCGC | |
| | A | |
| adhE-cs-down | CCGTTTATGTTGCCAGACAGCGCTACTGATTAAGC | 29 |
| | GGATTTTTTCGCTTTTTATTTGTTAACTGTTAATTGT | |
| | CCT | |
| adhE-del-down | CCGTTTATGTTGCCAGACAGCGCTACTGATTAAGC | 30 |
| | GGATTTTTTCGCTTTGCGCTCACACTCACTGTGAT | |
| | TTAC | |
| XZ-adhE-up | CATGCTAATGTAGCCACCAAA | 31 |
| XZ-adhE-down | TTGCACCACCATCCAGATAA | 32 |
| adhE-RBS5-up | ATAACTCTAATGTTTAAACTCTTTTAGTAAATCACAG | 33 |
| | TGAGTGTGAGCGCTTATCTCTGGCGGTGTTGAC | |
| adhE-kari-down | CCGTTTATGTTGCCAGACAGCGCTACTGATTAAGC | 34 |
| | GGATTTTTTCGCTTTTTAGATAACTTTTTTCTTCA | |
| pflB-CS-up | aaacgaccaccattaatggttgtcgaagtacgcagtaaataaaaaatcc | 35 |
| | aTGTGACGGAAGATCACTTCGCAG | |
| pflB-CS-down | CGGTCCGAACGGCGCGCCAGCACGACGACCGTC | 36 |
| | TGGGGTGTTACCCGTTTTTATTTGTTAACTGTTAAT | |
| | TGTCCT | |
| pflB-ilvD-up | aaacgaccaccattaatggttgtcgaagtacgcagtaaataaaaaatcc | 37 |
| | aatgcctaagtaccgttccgc | |
| pflB-ilvD-down | CGGTCCGAACGGCGCGCCAGCACGACGACCGTC | 38 |
| | TGGGGTGTTACCCGTTTttaaccccccagtttcgatttatc | |
| XZ-pflB-up600 | CTGCGGAGCCGATCTCTTTAC | 39 |
| XZ-pflB-down | CGAGTAATAACGTCCTGCTGCT | 40 |
| pflB-Pcs-up | aaacgaccaccattaatggttgtcgaagtacgcagtaaataaaaaatcc | 41 |
| | aTGTGACGGAAGATCACTTCGCA | |
| pflB-Pcs-down | CCCGCCATATTACGACCATGAGTGGTGGTGGCGG | 42 |
| | AACGGTACTTAGGCATTTATTTGTTAACTGTTAATT | |
| | GTCCT | |
| pflB-Pro-up | AAACGACCACCATTAATGGTTGTCGAAGTACGCAG | 43 |
| | TAAATAAAAAATCCATTATCTCTGGCGGTGTTGAC | |
| ilvD-Pro-down | cccgccatattacgaccatgagtggtggtggcggaacggtacttaggcat | 44 |
| | TGCTGACCTCCTGGTTTAAACGTACATG | |
| ilvD-YZ496-down | caaccagatcgagcttgatg | 45 |
| XZ-frd-up | TGCAGAAAACCATCGACAAG | 46 |
| Xz-frd-down | CACCAATCAGCGTGACAACT | 47 |
| frd-cs-up | GAAGGCGAATGGCTGAGATGAAAAACCTGAAAATT | 48 |
| | GAGGTGGTGCGCTATTGTGACGGAAGATCACTTC | |
| | GCA | |
| frd-cs-down | TCTCAGGCTCCTTACCAGTACAGGGCAACAAACA | 49 |
| | GGATTACGATGGTGGCTTATTTGTTAACTGTTAATT | |
| | GTCCT | |
| frd-M93-up | GAAGGCGAATGGCTGAGATGAAAAACCTGAAAATT | 50 |
| | GAGGTGGTGCGCTATTTATCTCTGGCGGTGTTGAC | |
| frd-leuDH-down | TCTCAGGCTCCTTACCAGTACAGGGCAACAAACA | 51 |
| | GGATTACGATGGTGGCTTAACGGCCGTTCAAAATA | |
| | TTTTTTTC | |
| ilvB pro-catup | ctgacgaaacctcgctccggcggggttttttgttatctgcaattcagtacTG | 52 |
| | TGACGGAAGATCACTTCGCA | |
| ilvB | tctgcgccggtaaagcgcttacgcgtcgatgttgtgcccgaacttgccatT | 53 |
| pro-catdown | TATTTGTTAACTGTTAATTGTCCT | |
| ilvB pro-up | ctgacgaaacctcgctccggcggggttttttgttatctgcaattcagtacTT | 54 |
| | ATCTCTGGCGGTGTTGAC | |
| ilvB pro-down | tctgcgccggtaaagcgcttacgcgtcgatgttgtgcccgaacttgccatA | 55 |
| | GCTGTTTCCTGGTTTAAAC | |
| ilvB pro-YZup | gttctgcgcggaacacgtatac | 56 |
| ilvB | ccgctacaggccatacagac | 57 |
| pro-YZdown | | |
| ilvG pro-catup | tgaactaagaggaagggaacaacattcagaccgaaattgaatttttttca | 58 |
| | TGTGACGGAAGATCACTTCGCA | |
| ilvG | ttcacaccctgtgcccgcaacgcatgtaccacccactgtgegccattcatT | 59 |
| pro-catdown | TATTTGTTAACTGTTAATTGTCCT | |
| ilvG pro-up | tgaactaagaggaagggaacaacattcagaccgaaattgaatttttttca | 60 |
| | TTATCTCTGGCGGTGTTGAC | |
| ilvG pro-down | ttcacaccctgtgcccgcaacgcatgtaccacccactgtgegccattcat | 61 |
| | AGCTGTTTCCTGGTTTAAACG | |
| ilvG pro-YZup | gcataagatatcgctgctgtag | 62 |
| ilvG p-YZdown | gccagttttgccagtagcac | 63 |
| ilvH*-cat-up | agaacctgattatgCGCCGGATATTATCAGTCTTACTCGA | 64 |
| | AAATGAATCATGTGACGGAAGATCACTTCGCA | |
| ilvH*-cat-down | TTCATCGCCCACGGTCTGGATGGTCATACGCGATA | 65 |
| | ATGTCGGATCGTCGGTTATTTGTTAACTGTTAATTG | |
| | TCCT | |
| ilvH*-mut-up | agaacctgattatgCGCCGGATATTATCAGTCTTACTCGA | 66 |
| | AAATGAATCAGaCGCGTTATtCCGCGTGATTGGC | |
| ilvH*-mut-down | CACACCAGAGCGAGCAACCTC | 67 |
| ilvH*-mutYZ-uP | atgagctggaaagcaaacttagc | 68 |

### Example 1: Knockout of Methylglyoxal Synthase Encoding Gene mgsA in ATCC 8739 Strain

Started from Escherichia coli ATCC 8739, a two-step homologous recombination method is used to knock out the methylglyoxal synthase encoding gene mgsA, and specific steps are as follows.

In a first step, a pXZ-CS plasmid DNA is used as a template, 2719 bp of a DNA fragment I is amplified by using primers mgsA-cs-up/mgsA-cs-down, and used for the first step of homologous recombination.

An amplification system is: Phusion 5× buffer (NewEngland Biolabs) 10 μl, dNTP (10 mM for each dNTP) 1 μl, DNA template 20 ng, primers (10 μM) 2 μl each, Phusion High-Fidelity DNA polymerase (2.5 U/μl) 0.5 μl, distilled water 33.5 μl, and a total volume is 50 μl.

Amplification conditions are 98° C. pre-denaturation for 2 minutes (1 cycle); 98° C. denaturation for 10 seconds, 56° C. annealing for 10 seconds, 72° C. extension for 2 minutes (30 cycles); and 72° C. extension for 10 minutes (1 cycle).

The above DNA fragment I is used for the first homologous recombination: firstly, a pKD46 plasmid (purchased from the Coil Genetic Stock Center (CGSC) of Yale University, CGSC #7739) is transformed into Escherichia coli ATCC 8739 by an electrotransformation method, and then the DNA fragment I is electrotransformed to the Escherichia coli ATCC 8739 with the pKD46.

Electrotransformation conditions are as follows: firstly, electrotransformation competent cells of the Escherichia coli ATCC 8739 with the pKD46 plasmid are prepared; 50 μl of the competent cells are placed on ice, and 50 ng of the DNA fragment I is added. The mixture was placed on ice for 2 minutes, and transferred into a 0.2 cm MicroPulser Electroporation Cuvette (Bio-Rad). The electroporation was carried with the MicroPulser (Bio-Rad) electroporation apparatus and the electric voltage was 2.5 kV. After electric shock, 1 ml of LB medium was quickly added into the electroporation cuvette, and transferred into a test tube after pipetting five times. The culture was incubated at 30° C. with shaking at 75 rpm for 2 hours. 200 μl of culture was spread onto a LB plate containing ampicillin (a final concentration is 100 μg/ml) and chloramphenicol (a final concentration is 34 μg/ml). After being cultured overnight at 30° C., colonies were verified with primers XZ-mgsA-up/XZ-mgsA-down, and a correct colony amplification product is a 3646 bp fragment. A correct single colony was selected, and named as Sval001.

In a second step, a genomic DNA of wild-type Escherichia coli ATCC 8739 is used as template, and 566 bp of a DNA fragment II is amplified with primers XZ-mgsA-up/mgsA-del-down. DNA fragment II is used for the second homologous recombination. Amplification conditions and system are the same as those described in the first step. The DNA fragment II is electrotransformed into strain Sval001.

Electrotransformation conditions are as follows: firstly, electrotransformation competent cells of the Sval001 with the pKD46 plasmid were prepared; 50 μl of the competent cells were placed on ice, and 50 ng of a DNA fragment II is added. The mixture was placed on ice for 2 minutes, and transferred into a 0.2 MicroPulser Electroporation Cuvette (Bio-Rad). The electroporation was carried with the MicroPulser (Bio-Rad) electroporation apparatus and the electric voltage was 2.5 kV. After electric shock, 1 ml of LB medium was quickly added into the electroporation cuvette, and transferred into a test tube after pipetting five times. The culture was incubated at 30° C. with shaking at 75 rpm for 4 hours. The culture was then transferred into LB medium containing 10% sucrose but without a sodium chloride (50 ml of a medium is loaded in 250 ml of a flask), and after being cultured for 24 hours, it is streak-cultured on an LB solid medium containing 6% sucrose without a sodium chloride. The correct clone was verified by colony PCR amplification with primers XZ-mgsA-up/mgsA-del-down, and a correct colony amplification product was 1027 bp. A correct single colony is then selected, and named as Sval002.

### Example 2: Knockout of Lactate Dehydrogenase Encoding Gene ldhA

Started from Sva1002, and a lactate dehydrogenase encoding gene ldhA is knocked out by a two-step homologous recombination method. Specific steps are as follows:

In a first step, a pXZ-CS plasmid DNA is used as a template, 2719 bp of a DNA fragment I is amplified by using primers ldhA-cs-up/ldhA-cs-down, and used for the first step of the homologous recombination. Amplification system and amplification conditions are the same as those described in Example 1. The DNA fragment I is electrotransformed to the Sval002.

The DNA fragment I is used for the first homologous recombination: firstly, a pKD46 plasmid is transformed into Escherichia coli Sval002 by an electrotransformation method, and then the DNA fragment I is electrotransformed into the Escherichia coli Sval002 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers XZ-ldhA-up/XZ-ldhA-down, and a correct PCR product should be 3448 bp. A correct single colony is picked, and named as Sval003.

In a second step, a DNA of wild-type Escherichia coli ATCC 8739 is used as a template, and 476 bp of a DNA fragment II is amplified with primers XZ-ldhA-up/ldhA-del-down. The DNA fragment II is used for the second homologous recombination. The DNA fragment II is electrotransformed into strain Sva1003.

Electrotransformation conditions and steps are the same as the second step method for the mgsA gene knockout described in Example 1. Colony PCR is used to verify clones using primers XZ-ldhA-up/XZ-ldhA-down, and a correct colony amplification product is 829 bp. A correct single colony is picked, and named as Sval004.

### Example 3: Knockout of Phosphoacetyl Transferase Encoding Gene Pta and Acetate Kinase Encoding Gene ackA

Started from Sval004, a two-step homologous recombination method is used to knock out a phosphoacetyl transferase encoding gene pta and an acetate kinase encoding gene ackA. Specific steps are as follows.

In a first step, a pXZ-CS plasmid DNA is used as a template, 2719 bp of a DNA fragment I is amplified by using primers ackA-cs-up/pta-cs-down, and used for the first step of the homologous recombination. Amplification system and amplification conditions are the same as those described in Example 1. The DNA fragment I is electrotransformed to the Sva1004.

The DNA fragment I is used for the first homologous recombination: firstly, a pKD46 plasmid is transformed into Escherichia coli Sva1004 by an electrotransformation method, and then the DNA fragment I is electrotransformed into the Escherichia coli Sva1004 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers XZ-ackA-up/XZ-pta-down, and a correct PCR product should be 3351 bp. A correct single colony is picked, and named as Sval005.

In a second step, a DNA of wild-type Escherichia coli ATCC 8739 is used as a template, and 371 bp of a DNA fragment II is amplified with primers XZ-ackA-up/ackA-del-down. The DNA fragment II is used for the second homologous recombination. The DNA fragment II is electrotransformed into strain Sva1005.

Electrotransformation conditions and steps are the same as the second step method for the mgsA gene knockout described in Example 1. Colony PCR is used to verify clones using primers XZ-ackA-up/XZ-pta-down, and a correct colony amplification product is 732 bp. A correct single colony is picked, and named as Sval006.

### Example 4: Knockout of Propionate Kinase Encoding Gene tdcD and Formate Acetyltransferase Encoding Gene tdcE

Started from Sval006, a two-step homologous recombination method is used to knock out the propionate kinase encoding gene tdcD and the formate acetyltransferase encoding gene tdcE. Specific steps are as follows.

In a first step, a pXZ-CS plasmid DNA is used as a template, 2719 bp of a DNA fragment I is amplified by using primers tdcDE-cs-up/tdcDE-cs-down, and used for the first step of the homologous recombination. Amplification system and amplification conditions are the same as those described in Example 1. The DNA fragment I is electrotransformed to the Sva1006.

The DNA fragment I is used for the first homologous recombination: firstly, a pKD46 plasmid is transformed into Escherichia coli Sval006 by an electrotransformation method, and then the DNA fragment I is electrotransformed into the Escherichia coli Sva1006 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers XZ-tdcDE-up/XZ-tdcDE-down, and a correct PCR product should be 4380 bp. A correct single colony is picked, and named as Sval007.

In a second step, a DNA of wild-type Escherichia coli ATCC 8739 is used as a template, and 895 bp of a DNA fragment II is amplified with primers XZ-tdcDE-up/tdcDE-del-down. The DNA fragment II is used for the second homologous recombination. The DNA fragment II is electrotransformed into strain Sva1007.

Electrotransformation conditions and steps are the same as the second step method for the mgsA gene knockout described in Example 1. Colony PCR is used to verify clones using primers XZ-tdcDE-up/XZ-tdcDE-down, and a correct colony amplification product is 1761 bp. A correct single colony is picked, and named as Sval008.

### Example 5: Knockout of Alcohol Dehydrogenase Gene adhE

Started from Sval008, a two-step homologous recombination method is used to knock out the alcohol dehydrogenase gene adhE. Specific steps are as follows.

In a first step, a pXZ-CS plasmid DNA is used as a template, 2719 bp of a DNA fragment I is amplified by using primers adhE-cs-up/adhE-cs-down, and used for the first step of the homologous recombination. Amplification system and amplification conditions are the same as those described in Example 1.

The DNA fragment I is used for the first homologous recombination: firstly, a pKD46 plasmid is transformed into Escherichia coli Sval008 by an electrotransformation method, and then the DNA fragment I is electrotransformed into the Escherichia coli Sva1008 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers XZ-adhE-up/XZ-adhE-down, and a correct PCR product should be 3167 bp. A correct single colony is picked, and named as Sva1009.

In a second step, a DNA of wild-type Escherichia co/1ATCC 8739 is used as a template, and 271 bp of a DNA fragment II is amplified with primers XZ-adhE-up/XZ-adhE-down. The DNA fragment II is used for the second homologous recombination. The DNA fragment II is electrotransformed into strain Sva1009.

Electrotransformation conditions and steps are the same as the second step method for the mgsA gene knockout described in Example 1. Colony PCR is used to verify clones using primers XZ-adhE-up/XZ-adhE-down, and a correct colony amplification product is 548 bp. A correct single colony is picked, and named as Sval010.

### Example 6: Integration of Acetohydroxy Acid Reductoisomerase Encoding Gene ilvC in Methylglyoxal Synthase Encoding Gene mgsA Site

Started from Sval010, an acetohydroxy acid reductoisomerase encoding gene ilvC from Escherichia coli is integrated into the methylglyoxal synthase encoding gene mgsA site through a two-step homologous recombination method. Specific steps are as follows.

In a first step, a cat-sacB fragment is integrated into the mgsA site of strain Sval010. PCR, integration, and verification of the cat-sacB fragment are exactly the same as the first step of the mgsA gene knockout in Example 1, and an obtained clone is named as Sval011.

In a second step, a DNA of wild-type Escherichia coli ATCC 8739 is used as a template, 1576 bp of a DNA fragment II is amplified by using primers mgsA-ilvC-up/mgsA-ilvC-down. The DNA fragment II is used for the second homologous recombination. The DNA fragment II is electrotransformed into strain Sval011.

Electrotransformation conditions and steps are the same as the second step method for the mgsA gene knockout described in Example 1. Colonies were PCR verified using primers XZ-mgsA-up/XZ-mgsA-down and sequenced, and a correct colony amplification product is 2503 by. A correct single colony is picked, and named as Sval012.

### Example 7: Regulation of Acetohydroxy Acid Reductoisomerase Encoding Gene ilvC

Started from Sval012, and an artificial regulatory element is used to regulate expression of the acetohydroxy acid reductoisomerase encoding gene ilvC integrated in methylglyoxal synthase encoding gene mgsA site. Specific steps are as follows.

In a first step, a pXZ-CS plasmid DNA is used as a template, 2719 bp of a DNA fragment I is amplified by using primers mgsA-Pcs-up/mgsA-Pcs-down, and used for the first step of the homologous recombination. Amplification system and amplification conditions are the same as those described in Example 1. The DNA fragment I is electrotransformed into the Sval012.

The DNA fragment I is used for the first homologous recombination: firstly, a pKD46 plasmid is transformed into Escherichia coli Sval012 by an electrotransformation method, and then the DNA fragment I is electrotransformed into the Escherichia coli Sval012 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers XZ-mgsA-up/ilvC-YZ347-down, and a correct PCR product should be 3482 bp. A correct single colony is picked, and named as Sva1013.

In a second step, a genomic DNA of M1-46 (Lu, et al., Appl Microbiol Biotechnol, 2012, 93:2455-2462) is used as a template, and 188 bp of a DNA fragment II is amplified by using primers mgsA-P46-up/ilvC-P46-down. The DNA fragment II is used for the second homologous recombination. The DNA fragment II is electrotransformed into strain Sval013.

Electrotransformation conditions and steps are the same as the second step method for the mgsA gene knockout described in Example 1. Colony PCR is used to verify clones using primers XZ-mgsA-up/ilvC-YZ347-down and sequenced, and a correct colony amplification product is 951 bp. A correct single colony is picked, and named as Sva1014.

### Example 8: Integration of Dihydroxy Acid Dehydratase Encoding Gene IND

Started from Sval014, a dihydroxy acid dehydratase encoding gene ilvD from Escherichia coli is integrated into the pyruvate formate lyase encoding gene pflB site and substitutes the pflB gene through a two-step homologous recombination method, namely the pflB gene is knocked out while the ilvD is integrated. Specific steps are as follows.

In a first step, a pXZ-CS plasmid DNA is used as a template, 2719 bp of a DNA fragment I is amplified by using primers pflB-CS-up/pflB-CS-down, and used for the first step of the homologous recombination. Amplification system and amplification conditions are the same as those described in Example 1. The DNA fragment I is electrotransformed into the Sval014.

The DNA fragment I is used for the first homologous recombination: firstly, a pKD46 plasmid is transformed into Escherichia coli Sval014 by an electrotransformation method, and then the DNA fragment I is electrotransformed into the Escherichia coli Sval014 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers XZ-pflB-up600/XZ-pflB-down, and a correct PCR product should be 3675 bp. A correct single colony is picked, and named as Sval015.

In a second step, a genomic DNA of Escherichia coli MG1655 (from ATCC, No. 700926) is used as a template, and 1951 bp of a DNA fragment II is amplified by using primers pflB-ilvD-up/pflB-ilvD-down. The DNA fragment II is used for the second homologous recombination. The DNA fragment II is electrotransformed into strain Sval015.

Electrotransformation conditions and steps are the same as the second step method for the mgsA gene knockout described in Example 1. Colonies were PCR verified using primers XZ-pflB-up600/XZ-pflB-down and sequenced, and a correct colony amplification product is 2907 by. A correct single colony is picked, and named as Sval016.

### Example 9: Regulation of Dihydroxy Acid Dehydratase Encoding Gene IND

Started from Sval016, and an artificial regulatory element is used to regulate expression of the dihydroxy acid dehydratase encoding gene ilvD integrated in the pyruvate formate lyase encoding gene pflB site. Specific steps are as follows.

In a first step, a pXZ-CS plasmid DNA is used as a template, 2719 bp of a DNA fragment I is amplified by using primers pflB-Pcs-up/pflB-Pcs-down, and used for the first step of the homologous recombination. Amplification system and amplification conditions are the same as those described in Example 1. The DNA fragment I is electrotransformed into the Sval016.

The DNA fragment I is used for the first homologous recombination: firstly, a pKD46 plasmid is transformed into Escherichia coli Sval016 by an electrotransformation method, and then the DNA fragment I is electrotransformed into the Escherichia coli Sval016 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers XZ-pflB-up600/ilvD-YZ496-down, and a correct PCR product should be 3756 bp. A correct single colony is picked, and named as Sval017.

In a second step, a genomic DNA of M1-93 (Lu, et al., Appl Microbiol Biotechnol, 2012, 93:2455-2462) is used as a template, and 189 bp of a DNA fragment II is amplified by using primers pflB-Pro-up/ilvD-Pro-down. The DNA fragment II is used for the second homologous recombination. The DNA fragment II is electrotransformed into strain Sval017.

Electrotransformation conditions and steps are the same as the second step method for the mgsA gene knockout described in Example 1. Colonies were PCR verified using primers XZ-pflB-up600/ilvD-YZ496-down and sequenced, and a correct colony amplification product is 1226 bp. A correct single colony is picked, and named as Sval018.

### Example 10: Regulation of Acetolactate Synthase Gene ilvBN

An artificial regulatory element M1-93 is used to regulate expression of an acetolactate synthase gene ilvBN through a two-step homologous recombination method. Specific steps are as follows.

In a first step, a pXZ-CS plasmid DNA is used as a template, 2719 bp of a DNA fragment I is amplified by using primers ilvB pro-catup/ilvB pro-catdown, and used for the first step of the homologous recombination. Amplification system and amplification conditions are the same as those described in Example 1.

The DNA fragment I is used for the first homologous recombination: firstly, a pKD46 plasmid is transformed into Escherichia coli Sval018 by an electrotransformation method, and then the DNA fragment I is electrotransformed into the Escherichia coli Sval018 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers ilvB pro-YZup/ilvB pro-YZdown, and a correct PCR product should be 2996 bp. A correct single colony is picked, and named as Sval019.

In a second step, a genomic DNA of M1-93 (Lu, et al., Appl Microbiol Biotechnol, 2012, 93:2455-2462) is used as a template, and 188 bp of a DNA fragment II is amplified by using primers ilvB pro-up/ilvB pro-down. The DNA fragment II is used for the second homologous recombination. The DNA fragment II is electrotransformed into strain Sval019.

Electrotransformation conditions and steps are the same as the second step method for the mgsA gene knockout described in Example 1. Colonies were PCR verified using primers ilvB pro-YZup/ilvB pro-YZdown and sequenced, and a correct colony amplification product is 465 bp. A correct single colony is picked, and named as Sval020.

### Example 11: Regulation of Acetolactate Synthase Gene ilvGM

An artificial regulatory element M1-93 is used to regulate expression of the acetolactate synthase gene ilvGM through a two-step homologous recombination method. Specific steps are as follows.

In a first step, a pXZ-CS plasmid DNA is used as a template, 2719 bp of a DNA fragment I is amplified by using primers ilvG pro-catup/ilvG pro-catdown, and used for the first step of the homologous recombination. Amplification system and amplification conditions are the same as those described in Example 1.

The DNA fragment I is used for the first homologous recombination: firstly, a pKD46 plasmid is transformed into Escherichia coli Sval020 by an electrotransformation method, and then the DNA fragment I is electrotransformed into the Escherichia coli Sva1020 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers ilvG pro-YZup/ilvG p-YZdown, and a correct PCR product should be 2993 bp. A correct single colony is picked, and named as Sval0121.

In a second step, a genomic DNA of M1-93 (Lu, et al., Appl Microbiol Biotechnol, 2012, 93:2455-2462) is used as a template, and 188 bp of a DNA fragment II is amplified by using primers ilvG pro-up/ilvG pro-down. The DNA fragment II is used for the second homologous recombination. The DNA fragment II is electrotransformed into strain Sval021.

Electrotransformation conditions and steps are the same as the second step method for the mgsA gene knockout described in Example 1. Colonies were PCR verified using primers ilvG pro-YZup/ilvG p-YZdown and sequenced, and a correct colony amplification product is 462 bp. A correct single colony is picked, and named as Sval022.

### Example 12: Mutation of Acetolactate Synthase Gene ilvH

A mutation is transferred into the ilvH gene so as to release feedback inhibition of L-valine through a two-step homologous recombination method. Specific steps are as follows.

In a first step, a pXZ-CS plasmid DNA is used as a template, 2719 bp of a DNA fragment I is amplified by using primers ilvH*-cat-up/ilvH*-cat-down, and used for the first step of the homologous recombination. Amplification system and amplification conditions are the same as those described in Example 1.

The DNA fragment I is used for the first homologous recombination: firstly, a pKD46 plasmid is transformed into Escherichia coli Sva1022 by an electrotransformation method, and then the DNA fragment I is electrotransformed into the Escherichia coli Sval022 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified using primers ilvH*-mutYZ-up/ilvH*-mut-down, and a correct PCR product should be 3165 bp. A correct single colony is picked, and named as Sval023.

In a second step, a DNA of wild-type Escherichia coli ATCC 8739 is used as a template, and 467 bp of a DNA fragment II is amplified by using primers ilvH*-mut-up/ilvH*-mut-down. The DNA fragment II is used for the second homologous recombination. The DNA fragment II is electrotransformed into strain Sva1023.

Electrotransformation conditions and steps are the same as the second step method for the mgsA gene knockout described in Example 1. Colonies were PCR verified using primers ilvH*-mutYZ-up/ilvH*-mut-down and sequenced, and a correct colony amplification product is 619 bp. A correct single colony is picked, and named as Sval024.

### Example 13: Fermentation and Production of L-Valine Using Recombinant Strain Sval024

A seed culture medium is formed by the following components (a solvent is water):

Glucose 20 g/L, corn syrup dry powder 10 g/L, KH₂PO₄ 8.8 g/L, (NH₄)₂SO₄ 2.5 g/L, and MgSO₄ 7H₂O 2 g/L.

The fermentation culture medium is most the same as the seed culture medium, and a difference is only that the glucose concentration is 50 g/L.

Anaerobic fermentation of Sval024 includes the following steps:

(1) Seed culture: a fresh clone on an LB plate is inoculated into a test tube containing 4 ml of the seed culture medium, and shake-cultured overnight at 37° C. and 250 rpm. Then, a culture is transferred to 250 ml of a triangular flask containing 30 ml of the seed culture medium according to an inoculum size of 2% (V/V), and seed culture solution is obtained by shake culture at 37° C. and 250 rpm for 12 hours, and used for fermentation medium inoculation.

(2) Fermentation culture: a volume of the fermentation culture medium in 500 ml of an fermenter is 250 ml, and the seed culture solution is inoculated into the fermentation culture medium according to an inoculum size of final concentration OD550=0.1, and fermented at 37° C. and 150 rpm for 4 days, to obtain fermentation solution. The neutralizer is 5M ammonia, the pH was controlled at 7.0. No air was sparged during the fermentation.

Analytical method: an Agilent (Agilent-1260) high performance liquid chromatograph is used to determine components in the fermentation solution after fermentation for 4 days. The concentrations of glucose and organic acid in the fermentation solution are determined by using an Aminex HPX-87H organic acid analytical column of Biorad Company. A Sielc amino acid analysis column primesep 100 250×4.6 mm is used for amino acid determination.

It is discovered from results that: strain Sval024 could produce 1.3 g/L of L-valine (L-valine peak corresponding to a position in FIG. 2 appears) with a yield of 0.31 mol/mol after 4 days fermentation under anaerobic conditions.

### Example 14: Cloning and Integration of Leucine Dehydrogenase Encoding Gene leuDH

Referring to the reported (Ohshima, T. et. al, Properties of crystalline leucine dehydrogenase from Bacillus sphaericus. The Journal of biological chemistry 253, 5719-5725 (1978)) sequence of a leuDH from Lysinibacillus sphaericus IFO 3525, a leuDH gene was codon optimized and chemically synthesized (an optimized sequence is as shown in a sequence number 69). During the synthesis, an M1-93 artificial regulatory element is added before the leuDH gene to initiate expression of the leuDH gene, and inserted into a pUC57 vector to construct a plasmid pUC57-M1-93-leuDH (gene synthesis and vector construction are completed by Nanjing Genscript Biotechnology Co., Ltd.). The M1-93 artificial regulatory element and the leuDH gene are integrated into the fumarate reductase encoding gene frd site in strain Sval024 through a two-step homologous recombination method and substitute the frd gene, namely the frd gene is knocked out while the leuDH is integrated. Specific steps are as follows.

In a first step, a pXZ-CS plasmid DNA is used as a template, 2719 bp of a DNA fragment I is amplified by using primers frd-cs-up/frd-cs-down, and used for the first step of the homologous recombination. Amplification system and amplification conditions are the same as those described in Example 1.

The DNA fragment I is used for the first homologous recombination: firstly, a pKD46 plasmid is transformed into Escherichia coli Sval024 by an electrotransformation method, and then the DNA fragment I is electrotransformed into the Escherichia coli Sva1024 with the pKD46.

Electrotransformation conditions and steps are the same as the first step method for the mgsA gene knockout described in Example 1. 200 μl of culture solution is spreaded onto a LB plate containing ampicillin (a final concentration is 100 μg/ml) and chloramphenicol (a final concentration is 34 μg/ml). After being cultured overnight at 30° C., colonies were PCR verified with primers XZ-frd-up/XZ-frd-down, and a correct PCR product should be 3493 bp. A correct single colony is picked, and named as Sval025.

In a second step, a pUC57-M1-93-leuDH plasmid DNA is used as a template, and 1283 bp of a DNA fragment II is amplified by using primers frd-M93-up/frd-leuDH-down. The DNA fragment II is used for the second homologous recombination. The DNA fragment II is electrotransformed into strain Sval025.

Electrotransformation conditions and steps are the same as the second step method for the mgsA gene knockout described in Example 1. Colonies PCR is used to verify clones using primers XZ-frd-up/XZ-frd-down and sequenced, and a correct colony amplification product is 2057 bp. A correct single colony is picked, and named as Sval026.

### Example 15: Fermentation and Production of L-Valine Using Recombinant Strain Sval026

Components and preparation of seed culture medium and fermentation culture medium are the same as those described in Example 13.

The fermentation is performed in 500 mL of a fermentation vessel, and a fermentation vessel and an analysis process are the same as the fermentation process and the analysis process of the Sval024 described in Example 13.

It is discovered from results that: the strain Sval026 could produce 1.8 g/L of L-valine (L-valine peak corresponding to a position in FIG. 2 appears) 0.56 mol/mol after 4 days fermentation under anaerobic conditions.

### Example 16: Integration of NADH-Dependent Acetohydroxy Acid Reductoisomerase Encoding Gene in Alcohol Dehydrogenase Gene adhE Site

An acetohydroxy acid reductoisomerase encoding gene kari is obtained according to a kari sequence from a Thermacetogenium phaeum strain reported in a literature (Brinkmann-Chen, S., Cahn, J K B & Arnold, FH Uncovering rare NADH-preferring ketol-acid reductoisomerases. Metab Eng 26, 17-22, doi: 10.1016/j.ymben.2014.08.003 (2014).) and through whole gene synthesis after being codon-optimized (an optimized sequence refers to a sequence 70). During the synthesis, the RBS5 artificial regulatory element is added in front of the kari gene and used to initiate expression of the kari gene, and inserted in the pUC57 vector, so a plasmid pUC57-RBS5-kari (gene synthesis and vector construction are completed by Nanjing Genscript Biotechnology Co., Ltd.) is constructed and obtained. The RBS5 artificial regulatory element and the kari gene are integrated into the adhE site of the alcohol dehydrogenase encoding gene in the strain Sval026 through a two-step homologous recombination method. Specific steps are as follows.

In a first step, a cat-sacB gene is integrated into the adhE gene site in the Sva1026, acquisition and purification of a fragment, integration of the first homologous recombination, and verification are the exactly same as the fragment and method used for the first step of the homologous recombination in the adhE gene knockout in Example 5, and clones obtained is named as Sval061 (Table 1).

In a second step, a pUC57-RBS5-kari plasmid DNA is used as a template, 1188 bp of a DNA fragment II is amplified with primers adhE-RBS5-up/adhE-kari-down. The DNA fragment II is used for the second homologous recombination. The DNA fragment II is electrotransformed into strain Sval061.

Electroporation conditions and steps are the same as the second step method for the mgsA gene knockout described in Example 1. Colonies were verified by PCR using primers XZ-adhE-up/XZ-adhE-down and sequenced, and a correct colony amplification product is 1636 by. A correct single colony is picked, and named as Sval062.

### Example 17: Knockout of NADPH-Dependent Acetohydroxy Acid Reductoisomerase Encoding Gene in mgsA Site

An NADPH-dependent acetohydroxy acid reductoisomerase encoding gene ilvC integrated in an mgsA site of a methylglyoxal synthase encoding gene is knocked out through a two-step homologous recombination method. Specific steps are as follows.

In a first step, a cat-sacB gene is integrated into the mgsA site to substitute the ilvC gene, acquisition and purification of a fragment, integration of the first homologous recombination, and verification are the exactly same as the fragment and method used for the first step of the homologous recombination in the mgsA gene knockout in Example 1, and a clone obtained is named as Sval063 (Table 1).

In a second step, the cat-sacB fragment is substituted with the fragment knocked out by using the mgsA, to obtain a strain knocked out by the mgsA gene and ilvC gene. Acquisition and purification of a fragment, integration of the second homologous recombination, and verification are the exactly same as the fragment and method used for the second step of the homologous recombination in the mgsA gene knockout in Example 1, and a clone obtained is named as Sval064.

### Example 18: Production of L-Valine Using Recombinant Strain Sval064

Components and preparation of seed culture medium and fermentation culture medium are the same as those described in Example 13.

The fermentation is performed in 500 mL of a fermentation vessel, a fermentation process and an analysis process are the same as the fermentation process and the analysis process of the Sval024 described in Example 13.

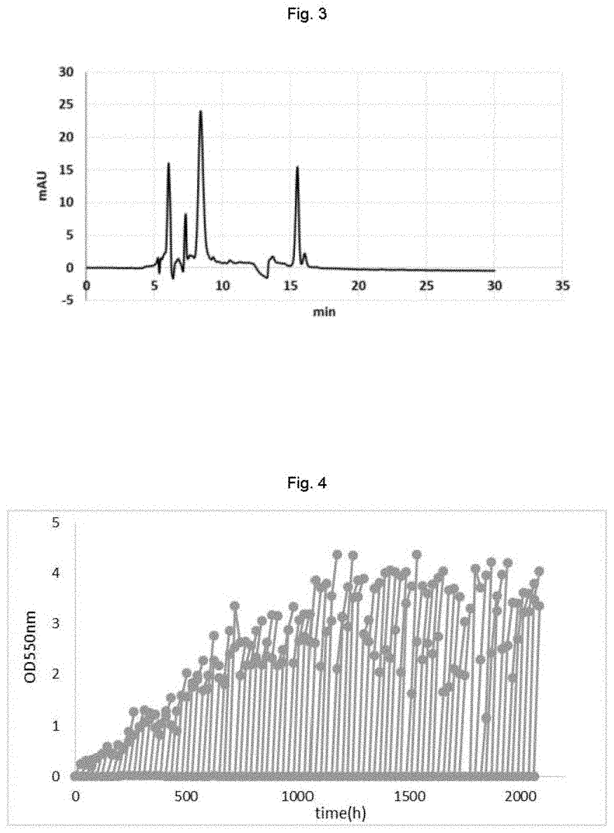

It is discovered from results that: the strain Sval064 could produce 2.0 g/L of L-valine (L-valine peak corresponding to a position in FIG. 2 appears) with a yield of 0.80 mol/mol (FIG. 3) after fermentation for 4 days under anaerobic conditions.

### Example 19: Construction of Recombinant Strain Sval065

Started from Sval064, cell growth and L-valine production capacity are synchronously improved through metabolic evolution.

Metabolic evolution was carried out in 500 mL fermentation vessel with 250 mL fermentation culture medium. The fermentative pH was controlled at 7.0 by using 5 Mammonia as neutralizer. Components and preparation method of fermentation culture medium used for the metabolic evolution are the same as those of the fermentation culture medium described in Example 16. Every 24 hours, fermentation solution is transferred into a new fermentation vessel and the initial OD550 is 0.1. After 105 generations of the evolution, a strain Sval065 is obtained (FIG. 4). The strain Sva1065 is preserved in China General Microbiological Culture Collection Center (CGMCC) with the preservation number CGMCC 19458.

### Example 20: Fermentation of Strain Sval065 to Produce L-Valine in 500 mL Fermentation Vessel

Components and preparation of a seed culture medium are the same as those described in Example 13.

The fermentation is performed in 500 mL of a fermentation vessel, and a fermentation culture medium is 250 ml. The fermentation culture medium is basically the same as the seed culture medium. A difference is that a glucose concentration is 100 g/L, and a neutralizer used is 5M ammonia, so that fermentative pH is controlled in 7.0.

It is discovered from results that: after fermented for 48 hours, strain Sval065 produced 45 g/LL-valine with a yield of 0.9 mol/mol, and impurities such as a heteroacid are not generated.

### Example 21: Production of L-Valine by Fermentation of Recombinant Strain Sval065 in 5 L Fermentation Vessel

Components, preparation and analytical method of a seed culture medium are the same as those described in Example 13. A fermentation culture medium is basically the same as the seed culture medium, and a difference is that a glucose concentration is 140 g/L.

The fermentation is performed in 5 L of a fermentation vessel (Shanghai Baoxing, BIOTECH-5BG) under anaerobic conditions, including the following steps:

(1) Seed culture: the seed culture medium in 500 ml of a triangular flask is 150 ml, and it is sterilized at 115° C. for 15 min. After cooling, recombinant Escherichia coli Sval045 are inoculated into the seed culture medium according to an inoculum size of 1% (V/V), and cultured at 37° C. and 100 rpm for 12 hours to obtain seed solution for inoculation of the fermentation culture medium.

(2) Fermentation culture: a volume of the fermentation culture medium in 5 L is 3 L, and it is sterilized at 115° C. for 25 min. The seed solution is inoculated into the fermentation culture medium according to an inoculum size of final concentration OD550=0.2, and cultured under anaerobic conditions at 37° C. for 3 days, and a stirring speed is 200 rpm, fermentation solution is obtained. The fermentation solution is all of substances in the fermentation vessel. No air was sparged during the fermentation.

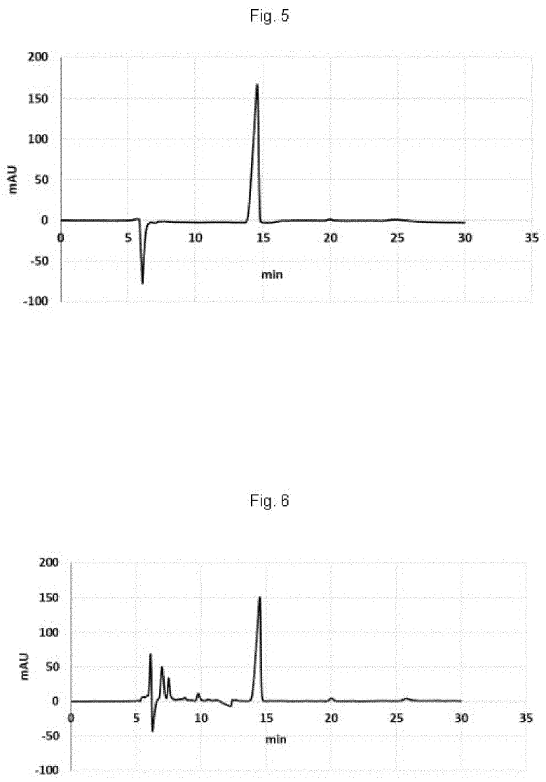

It is discovered from results that: after fermented for 48 hours, strain Sval065 produced 83 g/L L-valine with a yield of 0.92 mol/mol (0.6 g/g), and impurities such as a heteroacid are not generated (FIGS. 5-6).

What is claimed is:

1. A construction method of a recombinant microorganism for producing L-valine, comprising: transferring an amino acid dehydrogenase gene into a microorganism, and/or activating activity of a transhydrogenase in the microorganism, and/or activating activity of a NAD kinase in the microorganism, so that enhancing the activity of the transhydrogenase and/or the NAD kinase in the microorganism.

2. The construction method according to claim 1, wherein the method further comprises one or more of the following modifications (1)-(7) to the recombinant microorganism according to claim 1:
(1) knocking out a gene mgsA;
(2) knocking out a gene ldhA;
(3) knocking out genes pta and/or ackA;
(4) knocking out genes tdcD and/or tdcE;
(5) knocking out a gene adhE;
(6) knocking out genes frd and/or pflB; and
(7) enhancing activity of AHAS and/or ilvD;
preferably, the above items (7), (1), and (3)-(6) are selected for modification;
preferably, the above items (1)-(7) are selected for modification;
preferably, the item (6) is achieved by substituting the pflB gene of the microorganism itself with the ilvD gene;
preferably, the item (6) is achieved by substituting the frd gene of the microorganism itself with the leuDH gene; and
preferably, the item (1) is achieved by substituting the mgsA gene of the microorganism itself with the ilvC gene.

3. The construction method according to claim 1, wherein the microorganism is Escherichia coli; and more preferably, the microorganism is Escherichia coli ATCC 8739.

4. The construction method according to claim 1, wherein at least one regulatory element is used to activate or enhance activity of an encoding gene of the enzyme;
preferably, the regulatory element is selected from an M1-46 artificial regulatory element, an M1-93 artificial regulatory element or an M1-37 artificial regulatory element;
preferably, the M1-93 artificial regulatory element regulates encoding genes pntAB, ilvD, leuDH, ilvBN and ilvGM;
the M1-37 artificial regulatory element regulates an encoding gene yfjB; and
the M1-46 artificial regulatory element regulates an encoding gene ilvC.

5. The construction method according to claim 1, wherein one or more copies of the enzyme encoding gene and the regulatory element are integrated into a genome of the microorganism, or a plasmid containing the enzyme encoding gene is transferred into the microorganism;
preferably, transfer, mutation, knockout, activation or regulation of the enzyme gene is completed by a method of integrating into the genome of the microorganism;
preferably, the transfer, mutation, knockout, activation or regulation of the enzyme gene is completed by a homologous recombination method; and
preferably, the transfer, mutation, knockout, activation or regulation of the enzyme gene is completed by a two-step homologous recombination method.

6. A recombinant microorganism obtained by the construction method according to claim 1.

7. The construction method according to claim 1, wherein the construction method further comprises acquiring a recombinant microorganism for highly producing L-valine through metabolic evolution on the basis of the recombinant microorganism obtained by the construction method according to claim 1.

8. A recombinant microorganism, wherein a preservation number thereof is CGMCC 19456.

9. (canceled)

10. A method for producing L-valine, wherein the method comprises: (1) fermenting the recombinant microorganism according to claim 6; and (2) separating and harvesting L-valine; preferably, the fermentation is carried out under anaerobic conditions.

11. The construction method according to claim 1, wherein the construction method further comprises transferring an acetohydroxy acid reductoisomerase encoding gene into the microorganism so as to enhance activity of an acetohydroxy acid reductoisomerase; the acetohydroxy acid reductoisomerase encoding gene is preferably an ilvC gene.

12. The construction method according to claim 1, wherein the amino acid dehydrogenase gene is NADH-dependent.

13. The construction method according to claim 1, wherein the amino acid dehydrogenase gene is a leucine dehydrogenase gene.

14. The construction method according to claim 1, wherein the amino acid dehydrogenase gene is leuDH, the transhydrogenase is PntAB, and the NAD kinase is YfjB.

15. The construction method according to claim 2, wherein the AHAS is ilvBN, or ilvGM, or ilvIH; optionally, the activity of the ilvIH is enhanced by releasing feedback inhibition of valine to the ilvH, preferably, the ilvH gene is enhanced by mutation.

16. The construction method according to claim 2, wherein the item (7) is selected for modification.

17. The construction method according to claim 2, wherein the items (7) and (2) are selected for modification.

18. The construction method according to claim 2, wherein the items (7) and (6) are selected for modification.

19. The construction method according to claim 2, wherein the items (7), (2) and (5) are selected for modification.

20. The construction method according to claim 2, wherein the items (7), (2) and (6) are selected for modification.

21. A method for producing L-valine, wherein the method comprises: (1) fermenting the recombinant microorganism according to claim 8; and (2) separating and harvesting L-valine; preferably, the fermentation is carried out under anaerobic conditions.


SEQUENCE LISTING (70 sequences)

SEQ ID NO: 1 - DNA, 73 bp, Escherichia coli
gtaggaaagttaactacggatgtacattatggaactgacgactcgcactttgtgacggaagatcacttcgcag

SEQ ID NO: 2 - DNA, 75 bp, Escherichia coli
gcgtttgccacctgtgcaatattacttcagacggtccgcgagataacgctttatttgttaactgttaattgtcct

SEQ ID NO: 3 - DNA, 20 bp, Escherichia coli
cagctcatcaaccaggtcaa

SEQ ID NO: 4 - DNA, 20 bp, Escherichia coli
aaaagccgtcacgttattgg

SEQ ID NO: 5 - DNA, 70 bp, Escherichia coli
gcgtttgccacctgtgcaatattacttcagacggtccgcgagataacgctaagtgcgagtcgtcagttcc

SEQ ID NO: 6 - DNA, 72 bp, Escherichia coli
gtaggaaagttaactacggatgtacattatggaactgacgactcgcacttatggctaactacttcaatacac

SEQ ID NO: 7 - DNA, 75 bp, Escherichia coli
gcgtttgccacctgtgcaatattacttcagacggtccgcgagataacgctttaacccgcaacagcaatacgtttc

SEQ ID NO: 8 - DNA, 73 bp, Escherichia coli; identical to SEQ ID NO: 1

SEQ ID NO: 9 - DNA, 75 bp, Escherichia coli
agctgtgccagctgctggcgcagattcagtgtattgaagtagttagccatttatttgttaactgttaattgtcct

SEQ ID NO: 10 - DNA, 70 bp, Escherichia coli
gtaggaaagttaactacggatgtacattatggaactgacgactcgcacttttatctctggcggtgttgac

SEQ ID NO: 11 - DNA, 72 bp, Escherichia coli
agctgtgccagctgctggcgcagattcagtgtattgaagtagttagccatagctgtttcctggtttaaaccg

SEQ ID NO: 12 - DNA, 20 bp, Escherichia coli
cgcactacatcagagtgctg

SEQ ID NO: 13 - DNA, 73 bp, Escherichia coli
ttcaacatcactggagaaagtcttatgaaactcgccgtttatagcacaaatgtgacggaagatcacttcgcag

SEQ ID NO: 14 - DNA, 75 bp, Escherichia coli
agcggcaagattaaaccagttcgttcgggcaggtttcgcctttttccagattatttgttaactgttaattgtcct

SEQ ID NO: 15 - DNA, 20 bp, Escherichia coli
gataacggagatcgggaatg

SEQ ID NO: 16 - DNA, 20 bp, Escherichia coli
ctttggctgtcagttcacca

SEQ ID NO: 17 - DNA, 71 bp, Escherichia coli
agcggcaagattaaaccagttcgttcgggcaggtttcgcctttttccagatttgtgctataaacggcgagt

SEQ ID NO: 18 - DNA, 73 bp, Escherichia coli
aggtacttccatgtcgagtaagttagtactggttctgaactgcggtagtttgtgacggaagatcacttcgcag

SEQ ID NO: 19 - DNA, 75 bp, Escherichia coli
ggtcggcagaacgctgtaccgctttgtaggtggtgttaccggtgttcagattatttgttaactgttaattgtcct

SEQ ID NO: 20 - DNA, 20 bp, Escherichia coli
cgggacaacgttcaaaacat

SEQ ID NO: 21 - DNA, 20 bp, Escherichia coli
attgcccatcttcttgttgg

SEQ ID NO: 22 - DNA, 71 bp, Escherichia coli
ggtcggcagaacgctgtaccgctttgtaggtggtgttaccggtgttcagaaactaccgcagttcagaacca

SEQ ID NO: 23 - DNA, 73 bp, Escherichia coli
ccgtgattggtctgctgaccatcctgaacatcgtatacaaactgttttaatgtgacggaagatcacttcgcag

SEQ ID NO: 24 - DNA, 75 bp, Escherichia coli
cgcctggggcacgttgcgtttcgataatctttttcatacatcctccggcgttatttgttaactgttaattgtcct

SEQ ID NO: 25 - DNA, 21 bp, Escherichia coli
tgatgagctacctggtatggc

SEQ ID NO: 26 - DNA, 24 bp, Escherichia coli
cgccgacagagtaataggttttac

SEQ ID NO: 27 - DNA, 77 bp, Escherichia coli
cgcctggggcacgttgcgtttcgataatctttttcatacatcctccggcgttaaaacagtttgtatacgatgttcag

SEQ ID NO: 28 - DNA, 72 bp, Escherichia coli
ataactctaatgtttaaactcttttagtaaatcacagtgagtgtgagcgctgtgacggaagatcacttcgca

SEQ ID NO: 29 - DNA, 75 bp, Escherichia coli
ccgtttatgttgccagacagcgctactgattaagcggattttttcgctttttatttgttaactgttaattgtcct

SEQ ID NO: 30 - DNA, 74 bp, Escherichia coli
ccgtttatgttgccagacagcgctactgattaagcggattttttcgctttgcgctcacactcactgtgatttac

SEQ ID NO: 31 - DNA, 21 bp, Escherichia coli
catgctaatgtagccaccaaa

SEQ ID NO: 32 - DNA, 20 bp, Escherichia coli
ttgcaccaccatccagataa

SEQ ID NO: 33 - DNA, 70 bp, Escherichia coli
ataactctaatgtttaaactcttttagtaaatcacagtgagtgtgagcgcttatctctggcggtgttgac

SEQ ID NO: 34 - DNA, 70 bp, Escherichia coli
ccgtttatgttgccagacagcgctactgattaagcggattttttcgctttttagataacttttttcttca

SEQ ID NO: 35 - DNA, 73 bp, Escherichia coli
aaacgaccaccattaatggttgtcgaagtacgcagtaaataaaaaatccatgtgacggaagatcacttcgcag

SEQ ID NO: 36 - DNA, 75 bp, Escherichia coli
cggtccgaacggcgcgccagcacgacgaccgtctggggtgttacccgtttttatttgttaactgttaattgtcct

SEQ ID NO: 37 - DNA, 70 bp, Escherichia coli
aaacgaccaccattaatggttgtcgaagtacgcagtaaataaaaaatccaatgcctaagtaccgttccgc

SEQ ID NO: 38 - DNA, 74 bp, Escherichia coli
cggtccgaacggcgcgccagcacgacgaccgtctggggtgttacccgtttttaaccccccagtttcgatttatc

SEQ ID NO: 39 - DNA, 21 bp, Escherichia coli
ctgcggagccgatctctttac

SEQ ID NO: 40 - DNA, 22 bp, Escherichia coli
cgagtaataacgtcctgctgct

SEQ ID NO: 41 - DNA, 72 bp, Escherichia coli
aaacgaccaccattaatggttgtcgaagtacgcagtaaataaaaaatccatgtgacggaagatcacttcgca

SEQ ID NO: 42 - DNA, 75 bp, Escherichia coli
cccgccatattacgaccatgagtggtggtggcggaacggtacttaggcatttatttgttaactgttaattgtcct

SEQ ID NO: 43 - DNA, 70 bp, Escherichia coli
aaacgaccaccattaatggttgtcgaagtacgcagtaaataaaaaatccattatctctggcggtgttgac

SEQ ID NO: 44 - DNA, 78 bp, Escherichia coli
cccgccatattacgaccatgagtggtggtggcggaacggtacttaggcattgctgacctcctggtttaaacgtacatg

SEQ ID NO: 45 - DNA, 20 bp, Escherichia coli
caaccagatcgagcttgatg

SEQ ID NO: 46 - DNA, 20 bp, Escherichia coli
tgcagaaaaccatcgacaag

SEQ ID NO: 47 - DNA, 20 bp, Escherichia coli
caccaatcagcgtgacaact

SEQ ID NO: 48 - DNA, 72 bp, Escherichia coli
gaaggcgaatggctgagatgaaaaacctgaaaattgaggtggtgcgctattgtgacggaagatcacttcgca

SEQ ID NO: 49 - DNA, 75 bp, Escherichia coli
tctcaggctccttaccagtacagggcaacaaacaggattacgatggtggcttatttgttaactgttaattgtcct

SEQ ID NO: 50 - DNA, 70 bp, Escherichia coli
gaaggcgaatggctgagatgaaaaacctgaaaattgaggtggtgcgctatttatctctggcggtgttgac

SEQ ID NO: 51 - DNA, 77 bp, Escherichia coli
tctcaggctccttaccagtacagggcaacaaacaggattacgatggtggcttaacggccgttcaaaatatttttttc

SEQ ID NO: 52 - DNA, 72 bp, Escherichia coli
ctgacgaaacctcgctccggcggggttttttgttatctgcaattcagtactgtgacggaagatcacttcgca

SEQ ID NO: 53 - DNA, 75 bp, Escherichia coli
tctgcgccggtaaagcgcttacgcgtcgatgttgtgcccgaacttgccatttatttgttaactgttaattgtcct

SEQ ID NO: 54 - DNA, 70 bp, Escherichia coli
ctgacgaaacctcgctccggcggggttttttgttatctgcaattcagtacttatctctggcggtgttgac

SEQ ID NO: 55 - DNA, 70 bp, Escherichia coli
tctgcgccggtaaagcgcttacgcgtcgatgttgtgcccgaacttgccatagctgtttcctggtttaaac

SEQ ID NO: 56 - DNA, 22 bp, Escherichia coli
gttctgcgcggaacacgtatac

SEQ ID NO: 57 - DNA, 20 bp, Escherichia coli
ccgctacaggccatacagac

SEQ ID NO: 58 - DNA, 72 bp, Escherichia coli
tgaactaagaggaagggaacaacattcagaccgaaattgaatttttttcatgtgacggaagatcacttcgca

SEQ ID NO: 59 - DNA, 75 bp, Escherichia coli
ttcacaccctgtgcccgcaacgcatgtaccacccactgtgcgccattcatttatttgttaactgttaattgtcct

SEQ ID NO: 60 - DNA, 70 bp, Escherichia coli
tgaactaagaggaagggaacaacattcagaccgaaattgaatttttttcattatctctggcggtgttgac

SEQ ID NO: 61 - DNA, 71 bp, Escherichia coli
ttcacaccctgtgcccgcaacgcatgtaccacccactgtgcgccattcatagctgtttcctggtttaaacg

SEQ ID NO: 62 - DNA, 22 bp, Escherichia coli
gcataagatatcgctgctgtag

SEQ ID NO: 63 - DNA, 20 bp, Escherichia coli
gccagttttgccagtagcac

SEQ ID NO: 64 - DNA, 72 bp, Escherichia coli
agaacctgattatgcgccggatattatcagtcttactcgaaaatgaatcatgtgacggaagatcacttcgca

SEQ ID NO: 65 - DNA, 75 bp, Escherichia coli
ttcatcgcccacggtctggatggtcatacgcgataatgtcggatcgtcggttatttgttaactgttaattgtcct

SEQ ID NO: 66 - DNA, 74 bp, Escherichia coli
agaacctgattatgcgccggatattatcagtcttactcgaaaatgaatcagacgcgttattccgcgtgattggc

SEQ ID NO: 67 - DNA, 21 bp, Escherichia coli
cacaccagagcgagcaacctc

SEQ ID NO: 68 - DNA, 23 bp, Escherichia coli
atgagctggaaagcaaacttagc

SEQ ID NO: 69 - DNA, 1095 bp, Lysinibacillus sphaericus
atggaaatcttcaagtatatggaaaagtatgattatgaacaattggtattttgccaagacgaagcatctgggttaaaagcgattatcgctatccatgacacaacacttggaccagcattaggtggtgctcgtatgtggacctacgcgacagaagaaaatgcgattgaggatgcattaagattagcacgcgggatgacatataaaaatgcagctgctggtttaaaccttggcggtggaaaaacggtcattattggggacccatttaaagataaaaacgaagaaatgttccgtgctttaggtcgtttcattcaaggattaaacggtcgctatattaccgctgaagatgttggtacaaccgtaacagatatggatttaatccatgaggaaacaaattacgttacaggtatatcgccagcgtttggttcatcgggtaatccttcaccagtaactgcttatggcgtttatcgtggcatgaaagcagcggcgaaagaagcatttggtacggatatgctagaaggtcgtactatatcggtacaagggctaggaaacgtagcttacaagctttgcgagtatttacataatgaaggtgcaaaacttgtagtaacagatattaaccaagcggctattgatcgtgttgtcaatgattttggcgctacagcagttgcacctgatgaaatctattcacaagaagtcgatattttctcaccgtgtgcacttggcgcaattttaaatgacgaaacgattccgcaattaaaagcaaaagttattgctggttctgctaataaccaactacaagattcacgacatggagattatttacacgagctaggcattgtttatgcacctgactatgtcattaatgcaggtggtgtaataaatgtcgcggacgaattatatggctataatcgtgaacgagcgttgaaacgtgtagatggtatttacgatagtattgaaaaaatctttgaaatttccaaacgtgatagtattccaacatatgttgcggcaaatcgtttggcagaagaacgtattgctcgtgtagcgaaatcgcgtagtcagttcttaaaaaatgaaaaaaatattttgaacggccgttaa

SEQ ID NO: 70 - DNA, 999 bp, Thermacetogenium phaeum
atgaaaatctattacgatcaggacgcggatctgcaatatctggatggcaaaaccgtggctgttatcggctacggttcacagggccatgcgcagtcgcaaaatctgcgtgacagcggtgttaaagtggttgtcgcggatattccgagctctgaaaactggaaaaaagctgaagaagcgcagttccaaccgctgacggctgacgaagcagcccgcgaagcggatattatccagattctggtgccggatgaaaaacaagcagctctgtatcgtgaatcaatcgccccgaatctgcgcccgggcaaagcactggtgtttagccacggcttcaacattcactttaaacagatcgtgccgccgccggacgtcgatgtgtttatggtcgcaccgaaaggtccgggtcacctggtgcgtcgcatgtacgaagaaggcgccggtgttccgtctctggttgcagtcgaacaggactatagtggtcaagccctgaatctggcgctggcctacgcaaaaggcattggtgccacccgtgcaggcgtcatccagaccacgttcaaagaagaaaccgaaaccgacctgtttggtgaacaagccgtcctgtgcggcggtattaccgaactgatccgcgcaggcttcgacaccctggtggatgctggttatcagccggaaattgcgtactttgaatgtctgcatgaaatgaaactgattgttgacctgatctatgaaggcggtatttccaccatgcgttatagtatctccgacaccgctgaatacggcgatctgacgcgtggtaaacgcattatcaccgaagcgacgcgcgaagaaatgaagaaaattctgaaagaaatccaggatggcgtgttcgcccgtgaatggctgctggaaaaccaagtgggtcgcccggtttataatgccctgcgtcgcaaagaacagaaccacctgattgaaaccgtgggcgcacgtctgcgcggtatgatgccgtggctgaagaaaaaagttatctaa